(19) United States
(12) Patent Application Publication
(10) Pub. No.: US 2004/0106779 A1
(43) Pub. Date: Jun. 3, 2004

(54) Modified factor IX preparation

(76) Inventors: Douglas E. Bigler, Kankakee, IL (US); Sourav K. Kundu, Bourbonnais, IL (US); Holger Lind, Bourbonnais, IL (US); Stefan Schulte, Bourbonnais, IL (US)

Correspondence Address: Karen Guerrero, Aventis Behring L.L.C., 1020 First Avenue, King of Prussia, PA, 19406, US

(21) Appl. No.: 10/309,877

(22) Filed: Dec. 3, 2002

(51) Int. Cl. C07K 14/745

(52) U.S. Cl. 530/384

(57) ABSTRACT

The present invention relates to a modification of the anion exchange chromatography step in a Factor IX purification process. By the modification the presence of the protease, plasma hyaluronan binding protease, is decreased which minimizes the cleavage of Factor IX and enhances the yield of Factor IX in the purification process.

### FIELD OF THE INVENTION

This invention relates to the purification and stabilization of Factor IX, one of the proteins essential to the cascade of reactions, which accomplishes blood coagulation.

### BACKGROUND OF THE INVENTION

Factor IX is a globular protein which has a molecular weight of about 70,000 daltons and which, in a normal individual, is constantly produced in the liver and circulates in the blood and is involved in the coagulation process. The gene coding for Factor IX is located on the X chromosome. Functional Factor IX deficiencies can arise in different ways and cause hemophilia B. Some of the afflicted persons are known to have inherited an X chromosome with a complete deletion of the Factor IX gene. These severely affected persons may even produce antibodies to therapeutically injected Factor IX. Many hemophilia B patients are known to produce a Factor IX molecule with an altered amino acid sequence which results in molecules of partial or no coagulation activity. Some hemophilia B patients produce normal Factor IX, but in insufficient quantities to effect clotting within a normal time after injury.

To treat Hemophilia B, purified Factor IX can be administered to a patient. The purified Factor IX can be obtained from plasma or by recombinant methods. The purification of Factor IX from blood typically requires that it be separated from numerous other blood plasma proteins. Typically Factor IX is produced from cryoprecipitate-free plasma. This plasma fraction is produced by rapidly freezing whole plasma, allowing it to thaw slowly and collecting the separated supernatant. See Pool, J. G., et al. Nature, 203, 312 (1964). Many proteins, including Factor VII, precipitate out of the slowly thawing plasma and can be removed by centrifugation or filtration. Subsequently, the Factor IX-containing cryoprecipitate-free supernatant plasma is typically mixed with an anion exchange resin or gel leading to adsorption of the Factor IX along with other Prothrombin factors (i.e. Factors II, VII, and X, Protein C, Protein S and Protein Z) with similar binding properties, and also adsorption of other contaminating proteins. Initial fractionation with anion exchange resin particles takes advantage of the fact that Prothrombin factors (i.e. Factors II, VII, and X, Protein C, Protein S and Protein Z) are selectively adsorbed onto such resins owing to their negatively charged gamma carboxyglutamate residues. Typically, the bound Factor IX is washed, and then eluted from the anion exchange resin using a buffered salt solution of high molarity. The Factor IX-enriched eluate (contaminated with significant amounts of other proteins, including Prothrombin factors (i.e Factors II, VII, X, Protein C, Protein S and Protein Z) and other contaminating proteins is known as a prothrombin complex concentrate.

The above-mentioned anion exchange chromatography may also be preceded or replaced by other steps. For example, cryoprecipitate-free plasma, to which citrate has been added, can be treated with barium chloride causing precipitation of barium citrate on which Factor IX and certain other coagulation factors are bound. The proteins are isolated from the precipitate and then subjected to anion exchange chromatography. See, for example, Miletich, J. P. et al., J. Biol. Chem., 253(19), 6908-6916 (1978).

Alternatively, a second ion exchange resin step can be added. As disclosed in U.S. Pat. No. 4,447,416 (hereinafter the '416 patent), after anion exchange chromatography, the Factor IX fraction is subjected to ultrafiltration and diafiltration against 0.15 Molar NaCl buffered with 20 mM citrate, pH6, and then subjected to a second phase of ion exchange chromatography using a sulfated dextran resin. Factor IX is eluted from this second resin when the salt concentration reaches 0.8 Molar. The Factor IX, contained within a solution of high salt molarity, is then again subjected to ultrafiltration and diafiltration against physiologically acceptable 0.11 Molar NaCl, 20 mM citrate, pH 6.8, and then stored in lyophilized form. The method reported, however, leads to a Factor IX product in which less than 10% of the protein is Factor IX, more than 90% of the material consisting of contaminating protein species.

Michalski et. al., Vox Sang, 55, 202-210 (1988) discloses a Factor IX purifying strategy in which standard anion exchange chromatography of the prior art is followed by chromatography on a resin coated with heparin, a negatively charged mucopolysaccharide.

Since blood plasma contains many proteins which have similar physical properties and which are very difficult to separate from Factor IX, antibodies specific to Factor IX have been a valuable tool in attempting to isolate Factor IX in pure form. Following the technique of Koehler, G. and Milstein, C. (Nature, 256, 495-497 (1975)), Goodall, A. H. et al. identified monoclonal antibodies to Factor IX and used them in preparative immunoaffinity chromatography to create a high specific activity Factor IX. Blood, 59 (3), 664-670, (1982). Monoclonal antibody affinity techniques are very effective at separating Factor IX from other clotting factors and have become the preferred purifying method . However, in the present state of the art, Factor IX obtained by immunoaffinity chromatography is consistently contaminated with degraded forms of Factor IX, which cross react with Factor IX antibodies.

In order to produce the most high quality Factor IX, it is necessary to control decomposition of Factor IX throughout the preparative procedure and especially in the early steps of purification. Lack of a solution to this problem is consistently noted by the prior art. The present invention relates to stabilizing Factor IX against degradation by decreasing the presence of a particular protease, which specifically targets Factor IX in the purification process.

### SUMMARY OF THE INVENTION

In accordance with the present invention, there is provided a process for purifying and stabilizing Factor IX derived from human blood plasma. The improvement entails separating the Factor IX from a protease found in plasma to decrease the extent of Factor IX degradation. The protease is described in the literature as plasma hyaluronan binding protease (PHBP) or Factor VII activating protease (FSAP) ((Choi-Miura et al., J. Biochem. 119,1157-1165 (1996) and Roemisch et al., Blood Coag. and Fibrin. 10, 471-479 (1999)) In one aspect, the present invention involves increasing Factor IX stability without sacrificing yield by decreasing the amount of protease purified along with Factor IX. And in particular, the present invention entails decreasing the ratio of Factor IX to a particular protease, plasma hyaluronan binding protease (PHBP), that affects the degradation of Factor IX. In this manner, the extent of Factor IX degradation in the final purified product is decreased compared with Factor IX purified by other methods. The method entails (a) obtaining a crude Factor IX protein-containing sample, (b) contacting the sample with an ionic exchange chromatographic medium so that said Factor IX protein binds; and then eluting said Factor IX protein from the chromatographic medium with a low ionic strength solution comprising between about 0.35M to 0.4M salt. In this manner; Factor IX is separated from the protease thereby increasing the stability of Factor IX. Exemplary salts include NaCl or LiCl.

The purification scheme may include other steps, including affinity chromatography steps, such as monoclonal antibody affinity chromatography, or a viral reduction steps, such as high salt treatment, pasteurization or nanofiltration.

### BRIEF DESCRIPTION OF THE FIGURES

FIG. 1 compares the stability of a Factor IX preparation at 2, 6 and 24 hours prepared by the method of the present invention versus a batch elution method by non-reduced Western blots.

FIG. 2 shows Western blots of Factor IX preparations prepared by the method of the present invention versus a batch elution method.

FIGS. 3 and 4 show Western blots of Factor IX preparations after the monoclonal affinity chromatography step prepared by the method of the present invention versus a batch elution method which shows the presence of substantially more Factor IX degradation in the batch elution method.

### DETAILED DESCRIPTION OF THE INVENTION

The production of Factor IX for therapeutic use from plasma typically begins with blood plasma which is subjected to freezing. The frozen plasma is then slowly thawed at which point clotting Factor VIII and certain other proteins can be recovered as a cryoprecipitate. Factor IX and other proteins move into the soluble supernatant.

This Factor IX-containing plasma fraction is then typically subjected to adsorption on an anion exchange resin. After washing the resin particles extensively with a dilute salt solution to remove unbound or weakly binding proteins, a high molarity salt solution is usually used to elute Factor IX which is collected in a fraction known as the “prothrombin complex concentrate” because it also contains significant amounts of the other vitamin K-dependent clotting factors (Factors II, VII, X, Protein C, Protein S and Protein Z) and an unidentified protease capable of selectively degrading Factor IX.

We investigated the nature of the Factor IX degradation products observed during purification of Factor IX. Purified preparations of Factor IX showed on SDS-PAGE the intact Factor IX molecule migrating at approximately 70 kd and minor Factor IX bands at 50 kd and 12 kd. (S. A. Limentani et al., Thrombosis and Hemostasis 73 (4), 584-91 (1995)) These minor Factor IX bands were analyzed with protein-chemical techniques and identified as degradation products of Factor IX generated by a single cleavage within the protease domain of Factor IX after residue 318 (i.e. cleavage between arginine (318) and serine (319)). Thus, the 50 kd Factor IX band starts with the N-terminal sequence Y-N-S-G and ends with the sequence H-K-G-R. The 12 kd band starts with the N-terminal sequence S-A-L-V and ends with the sequence T-K-L-T. This cleavage of Factor IX has not been described previously in the literature and we were interested in identifying and purifying the corresponding protease causing this cleavage. Using intact Factor IX as a substrate we were able to establish an assay to measure the activity of the protease. Using this assay a purification of the protease was developed. Protein-sequencing of the N-terminus of the purified protease identified the enzyme as a protease which had previously been identified as plasma hyaluronan-binding protein (PHBP) (Choi-Miura et al., J. Biochem. 119, 1157-1165 (1996)).

It is therefore a key aspect of the present invention to decrease the presence of PHBP and increase the ratio of Factor IX to the protease in a biological sample by modifying the early stages of Factor IX purification (such as the anion exchange chromatography step) commonly in practice today. In the modified anion exchange chromatography step, a crude Factor IX protein-containing sample is obtained. Then the sample is contacted with an ionic exchange chromatographic medium so that the Factor IX protein binds to the chromatographic medium, and the Factor IX protein is eluted from the chromatographic medium with a low ionic strength solution comprising between preferably about 0.35M to 0.4M salt. At 0.5M salt there appeared to be some contamination with PHBP. However at between 0.35M to 0.4M salt, the extent of contamination with the protease was decreased while at the same time the yield of Factor IX was not adversely affected compared with elutions performed at higher salt. At lower salt concentrations (0.3M) there was a substantial decrease in the yield of Factor IX. Result details are provided in the Examples.

We also performed stability studies comparing the Factor IX stability after elution at 0.4M salt with that of Factor IX eluted under high salt conditions. As shown in the Examples, the Factor IX eluted under low salt conditions was substantially more stable than the high salt eluted Factor IX.

Once the anion exchange chromatography procedure is completed, additional separation procedures can be used to further purify the Factor IX. Examples of such additional separation procedures include the use of chromatography on an agarose gel to which heparin groups have been attached, cation exchange on a sulfated dextran gel, or immunoaffinity chromatography in which the stationary phase of the separation (purification) system consists of Factor IX-specific antibodies. Additional purification procedures may further include salt fractionation, ultrafiltration, nanofiltration and pasteurization.

The Factor IX purification may employ cryoprecipitation of blood plasma to remove proteins such as Factor VIII, followed by anion exchange chromatography which takes advantage of the specific adsorbability of vitamin K-dependent clotting factors followed by elution at low salt, followed by immunoaffinity chromatography using monoclonal antibodies, the last mentioned separation process having great specificity for Factor IX compared with other protein species. Additional steps may include an ultrafiltration step, one or more viral reduction steps, pasteurization and nanofiltration. Furthermore, a chromatographic step can be performed to remove impurities from the immunoaffininty chromatography step. The purification steps may be performed at room temperature or a lower temperatures.

And in particular, Factor IX may be produced from pooled human blood plasma, either recovered plasma units or source plasma. A particular purification process is described below. After removal of the cryoprecipitated fraction, the remaining supernatant fluid is fractionated by ion exchange chromatography using DEAE Sephadex resin. The eluate may further be purified by immunoaffinity chromatography through a column containing matrix-bound murine Factor IX monoclonal antibody. The monoclonal antibody-bound Factor IX protein is eluted from the immunoaffinity matrix using using sodium thiocyanate solution, and the eluate is incubated in 3 M sodium thiocyanate elution buffer for at least 3 hours for virus inactivation. The incubated filtrate is ultrafiltered to remove thiocyanate, pasteurized in the presence of sucrose and glycine at sixty degrees Centigrade for 10.5 hours, and filtered through two sequential membranes, each with an approximate molecular weight cutoff of 100,000 (100 kDa). Dual nanofiltration allows passage of Factor IX, while retaining many viruses. The filtrate is then concentrated, and adsorbed onto and then eluted from an additional ion exchange resin (aminohexyl-Sepharose gel; AH Sepharose) to remove trace residual murine monoclonal antibodies. The eluate is dialyzed or diafiltered to achieve the approximate excipient concentrations. The product is filled into vials and lyophilized.

Naturally, there can be substantial modification of the above described protocol using steps well known to those of ordinary skill in the art.

Throughout the purification process measurements of Factor IX activity may be performed using assays well known in the art. For example, some of the assays which are presently used include a two stage clotting assay, (Leibman, H. A. et al., Proc. Natl. Acad. Sci., USA, 82, 3879-3883 (1985)); an assay based on the single stage activated partial thromboplastin time (“APTT”), Smith, K. J. et al., Blood, 72,1269-1277 (1988); and assays which are modifications of the APTT test, for example, Jenny, R. et al., Preparative Biochemistry, 16, 227-245 (1986). Assays based on antigenic potency of Factor IX have also been proposed as a measure of purity (Smith, K. J. et al., Thromb. Haemostas., 58, 349 (1987)). Whereas, measurements of protease activity may be measured as provided in Roemisch et al. or Choi-Muira et al. (supra). Additionally, the presence and extent of Factor IX degradation may be measured by standard techniques such as sodium dodecylsulfate polyacrylamide gel electrophoresis (SDS-PAGE), and Western blotting of electrophoresed Factor IX-containing samples.

### EXAMPLES

### Example 1

### DEAE Adsorption

All processing described below was performed between 2-8° C. 1.5g dry DEAE Sephadex A-50 was swelled and added per liter of cryo-poor plasma and agitated for 1 hour. The DEAE resin was separated from the resin/ plasma suspension by being pumped into a <5 cm bed height column fitted with an agitator in place of the column flow adapter. A second pump connected to the column outlet simultaneously transferred plasma to a holding tank for downstream fractionation.

The adsorbed DEAE resin was resuspended with agitation in a minimal volume of wash buffer (10 mM sodium citrate, 0.18M NaCl, pH 7.0); and continuously washed with 1L of wash buffer per liter plasma as wash was pumped from bottom of column to waste at a rate equivalent to the addition of wash buffer.

### Example 2

### DEAE Chromatography

The washed and adsorbed DEAE resin was resuspended in wash buffer, pumped into a secondary 10-20 cm bed height column and packed. The column was eluted with elution buffer (between about 0.35-0.40M NaCl, 10 mM sodium citrate, pH 7.0). Eluate was collected at the start of the effluent conductivity peak and ending at the end of the UV peak. The DEAE eluate was either frozen or immediately processed further.

In these experiments it was demonstrated that transferring the washed DEAE to a chromatography column and eluting it with 0.35M or 0.40M NaCl resulted in a significant increase in the ratio of Factor IX (FIX) to PHBP, while maintaining yield values equivalent to those obtained with the current process (Table 1). As shown the ratio increased about 40 fold at 0.4 M NaCl and was even higher (about 100 fold) at 0.3 M. In contrast the yield of Factor IX from either the DEAE eluate or after the immunoaffinity chromatography step (procedure outlined in Example 3) remained substantially the same at these concentrations of salt.

TABLE 1

| Process | DEAE FIX:PHBP | | DEAE Eluate Yield | | MAb Eluate Yield | |
|---|---|---|---|---|---|---|
| 2.0M DEAE Batch Elution | 7:1 | (n = 2) | 792 | U/L (historical) | 607 | U/L (n = 463) |
| 0.4M DEAE Column Elution | 317 | (n = 9) | 910 | U/L (n = 10) | 715 | U/L (n = 3) |
| 0.35M DEAE Column Elution | 648:1 | (n = 2) | 1001 | U/L (n = 2) | 687 | U/L (n = 2) |

It was also observed that if the NaCl concentration was decreased further to 0.3M that the recovery of Factor IX was substantially decreased as shown in Table 2. Table 2 shows that 0.35M NaCl gives a significant (2 fold) reduction in PHBP without sacrifice of yield compared to elution with 0.4M NaCl. Whereas when the anion exchange column is eluted with 0.3M NaCl results show a 4 fold reduction in PHBP, but the procedure results in 25% and 35% less yield at the DEAE and immunoaffinity steps respectively compared to elution with 0.4M salt.

TABLE 2

| Process | DEAE FIX:PHBP | | DEAE Eluate Yield | | MAb Eluate Yield | |
|---|---|---|---|---|---|---|
| 2.0M DEAE Batch Elution | 7:1 | (n = 2) | 792 | U/L (historical) | 607 | U/L (n = 463) |
| 0.5M DEAE Column Elution | 50 | (n = 1) | 725 | (n = 1) | NA | |
| 0.4M DEAE Column Elution | 317 | (n = 9) | 910 | U/L (n = 10) | 715 | U/L (n = 3) |
| 0.35M DEAE Column Elution | 648:1 | (n = 2) | 1001 | U/L (n = 2) | 687 | U/L (n = 2) |
| 0.3M DEAE Column Elution | 1250 | (n = 2) | 684 | (n = 2) | 463 | (n = 2) |

Also compared in Table 2 was the ratio of Factor IX to PHBP during elution with 0.5M NaCl versus 0.4M NaCl. The 0.5M salt elution conditions eluted approximately 6 fold the amount of PHBP compared with 0.4M salt elution conditions.

### Example 3

### Monoclonal Antibody (MAb) Chromatography

An anti FIX-monoclonal antibody column (sized 62.5 ml resin per L plasma, ˜20 cm bed height) was: equilibrated with 2 column volumes TE Buffer (50 mM TRIS, 10 mM EDTA, pH 8.0); washed with 2 column volumes LLTE Buffer (10 mM EDTA, 50 mM TRIS, 0.5M lithium chloride, 0.1M lysine, pH 8.0) and 2 column volumes TE Buffer; then eluted with 2 column volumes of elution buffer (50 mM TRIS, 10 mM EDTA, 3M sodium thiocyanate, pH 8.0). 1.1 column volumes of eluate was collected at the start of the effluent UV Peak. The eluate was incubated for 3 hours. The flow rate was 2 column volumes per hour except for elution at 1.4 column volumes per hour. The incubated eluate was immediately processed further.

### Example 4

### Ultrafiltration

The incubated eluate was then: concentrated via 10-30K molecular weight cutoff ultrafiltration to 55% of initial volume; diafiltered with 5 exchanges of G-25 Buffer (5 mM histidine, 50 mM sodium chloride, pH 7.0); and concentrated to 10% of initial volume. The diafiltered, concentrated eluate was either frozen or immediately processed further.

### Example 5

### Pasteurization

2.36M sucrose, 0.68M glycine, 0.13M calcium chloride were dissolved in the diafiltered eluate and the stabilized eluate was transferred to 1L glass bottles. The bottled, stabilized, concentrated eluate was submersed in a water bath and heated to 60° C. for 10.5 hours.

### Example 6

### Nanofiltration

The pasteurized eluate was diluted in G-25 Buffer, diafiltered with 5 exchanges G-25 Buffer, and passed through two nanofilters in series. The type of nanofilters that can be used are Asahi Kasei Planova P-15 or P-20, Millipore Viresolve NFP or similar. Alternatively, an ultrafiltration membrane such as Millipore YM-100 membrane can also be utilized for virus reduction purposes. The nanofiltered sample was either frozen or immediately processed further.

### Example 7

### AH Sepharose Chromatography

This chromatography step was performed using an AH Sepharose column with a volume of 4 ml resin per 5000 Units FIX and ˜7 cm bed height. The flow rate was 10 column volumes per hour except for elution, which was at 5 column volumes per hour. The column was equilibrated with 10 column volumes of AH equilibration Buffer (10 mM histidine, 5 mM lysine, 150 mM sodium chloride pH 7.0). The nanofiltered sample was loaded and washed with 10 column volumes of AH equilibration Buffer. The column was eluted with AH elution Buffer (50 mM calcium chloride in AH equilibration Buffer, pH 7.0). The eluate was collected beginning with the UV peak and ending at 5% above the UV baseline. The AH Sepharose eluate was dialyzed or diafiltered in Factor IX AH-diafiltration Buffer (10 mM histidine, 3% mannitol, 67 mM sodium chloride, 0.0075% Tween 80 (optional), pH 7.0). The diafiltered AH Sepharose eluate was diluted to 116 FIX U/ml in diafiltration Buffer and sterile filtered. The sterilized sample was filled in vials having various dosages and lyophilized.

### Example 8

### Stability Studies

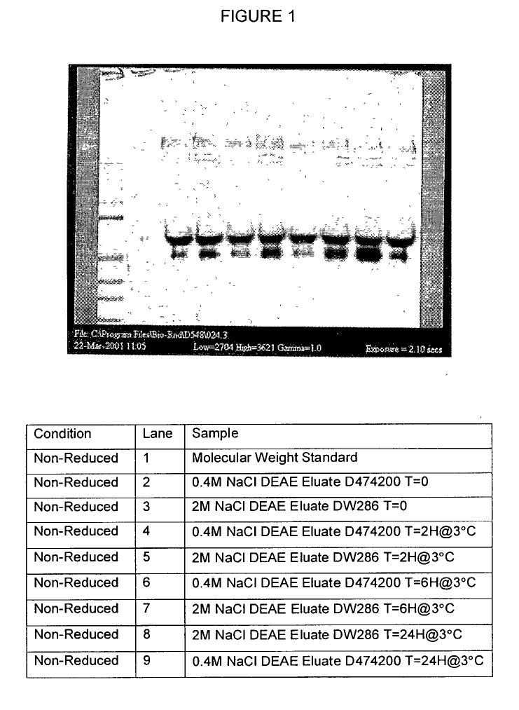

A study was performed comparing the FIX protein stability of 0.4M NaCl DEAE column eluate to 2M NaCl DEAE batch eluate at 3° C. and 20° C. as measured by FIX Western Blot demonstrated improved stability at 2,6, and 24 hours (FIG. 1). The samples of 2M NaCl batch eluate showed a more prominent degradation band on the Western Blot, demonstrating that reduction of PHBP in the DEAE eluate resulted in much less degradation of FIX protein.

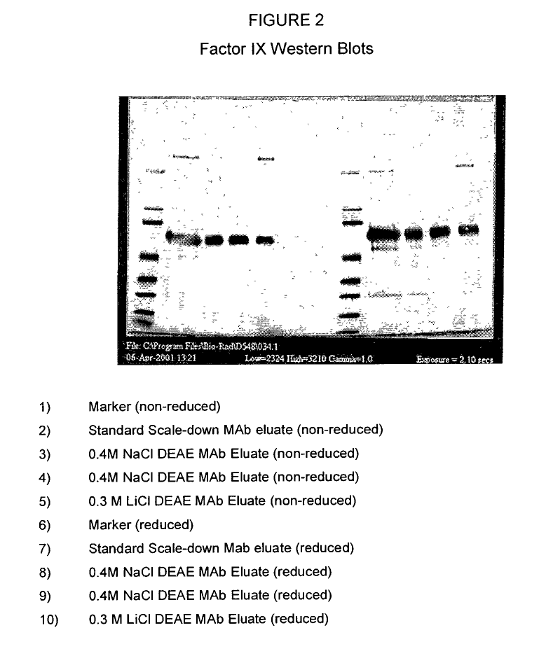

Non-reduced Western Blots of scale-down MAb eluates derived from 0.4M NaCl DEAE column eluates consistently appear as a prominent single band with little or no higher and lower molecular weight bands, in contrast to MAb eluates derived from 2M NaCl DEAE batch eluates, which show additional high and low molecular weight bands. This indicates that an increase in intermediate Factor IX protein stability and purity is obtainable by using 0.4M NaCl DEAE column elution instead of 2M NaCl batch elution (FIG. 2).

### Example 9

### Substitution of LiCi for NaCl

Substitution of Lithium Chloride for Sodium Chloride in the DEAE column elution might further improve Factor IX protein stability was also evaluated. The FIX:PHBP ratio for 0.3M LiCl DEAE eluates was 3933 (n=4) compared to 1250 (n=2) for 0.3M NaCl DEAE eluates. (Table 3).

TABLE 3

| DEAE Eluant | Plasma Lot | DEAE Eluate Yield | DEAE FIX:PHBP |
|---|---|---|---|
| 0.3M LiCl | XPX106 | 638 U/L Plasma | 4773 |
| 0.3M LiCl | XPX106 | 909 U/L Plasma | 4115 |
| 0.3M LiCl | ZPX046 | 756 U/L Plasma | 4813 |
| 0.3M NaCl | ZPX046 | 646 U/L Plasma | 846 |
| 0.3M LiCl | XPX159 | 616 U/L Plasma | 2032 |
| 0.3M NaCl | XPX159 | 721 U/L Plasma | 1653 |

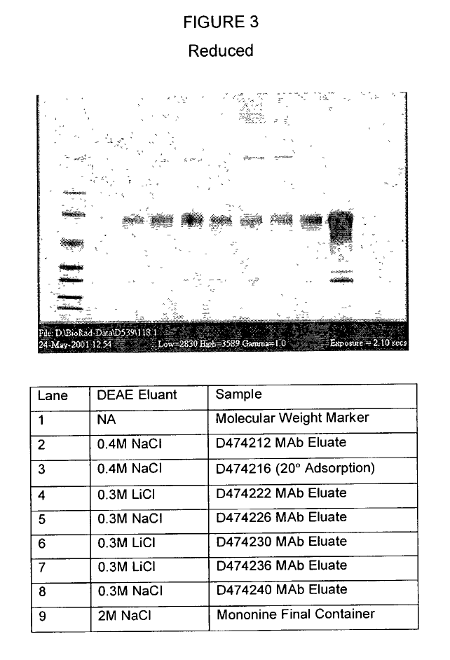

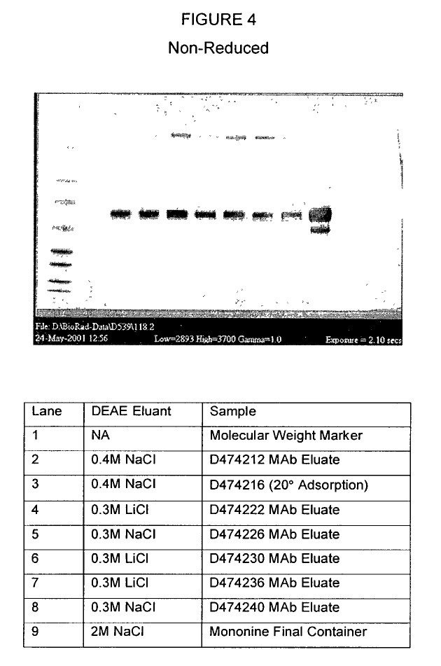

Western Blots of MAb eluates derived from LiCi DEAE column eluates consistently show a prominent high molecular weight band that is absent or faint in those derived from NaCl DEAE column eluates. This might indicate a decreased intermediate Factor IX protein purity associated with the use of LiCl in DEAE column chromatography. There is however no visible difference in the degradation band between LiCl and NaCl eluted material (FIGS. 3,4).

Scale-up of the modification from 1:800 to 1:200 of full-scale (1 L to 4L of plasma) maintained the yield and protease-reduction values found at the smaller scale. (Table 4)

TABLE 4

| Scale | DEAE FIX:PHBP | DEAE Eluate Yield |
|---|---|---|
| 1:800 | 254:1 (n = 7) | 870 U/L (n = 7) |
| 1:200 | 410:1 (n = 3) | 897 U/L (n = 3) |

Although the invention herein has been described with reference to particular embodiments, it is to be understood that these embodiments are merely illustrative of various aspects of the invention. Thus, it is to be understood that numerous modifications may be made in the illustrative embodiments and other arrangements may be devised without departing from the spirit and scope of the invention.

1. A method for increasing Factor IX stability without sacrificing Factor IX yield by separating Factor IX from plasma hyaluronan binding protease, said method comprising:
a. obtaining a crude Factor IX protein-containing sample;
b. contacting said sample with an ionic exchange chromatographic medium so that said Factor IX protein binds; and
c. eluting said Factor IX protein from the chromatographic medium with a low ionic strength solution comprising between about 0.35M to 0.4M salt; whereby said Factor IX is separated from the plasma hyaluronan binding protease thereby increasing the stability of Factor IX.

2. The method of claim 1, further comprising an affinity chromatography purification step.

3. The method of claim 2, wherein said affinity chromatography purification step is monoclonal antibody chromatography.

4. The method of claim 1, further comprising a virus reduction step.

5. The method of claim 3, wherein said virus reduction step comprises a pasteurization or nanofiltration step.

6. The method of claim 1, wherein said salt is NaCl or LiCl.

7. A method for increasing the ratio of Factor IX to plasma hyaluronan binding protease, said method comprising:
a. obtaining a crude Factor IX protein-containing sample;
b. contacting said sample with an ionic exchange chromatographic medium so that said Factor IX protein binds; and
c. eluting said Factor IX protein from the chromatographic medium with a low ionic strength solution comprising between about 0.35M to 0.4M salt;
whereby said Factor IX is separated from said protease thereby increasing the ratio of Factor IX to said plasma hyaluronan binding protease.

8. The method of claim 7, further comprising an affinity chromatography purification step.

9. The method of claim 8, wherein said affinity chromatography purification step is monoclonal antibody chromatography.

10. The method of claim 7, further comprising a virus reduction step.

11. The method of claim 10, wherein said virus reduction step comprises a pasteurization or nanofiltration step.

12. The method of claim 8, wherein said salt is NaCl or LiCl.

13. A method for decreasing the extent of Factor IX degradation, said method comprising:
a. obtaining a crude Factor IX protein-containing sample;
b. contacting said sample with an ionic exchange chromatographic medium so that said Factor IX protein binds; and
c. eluting said Factor IX protein from the chromatographic medium with a low ionic strength solution comprising between about 0.35M to 0.4M salt; whereby said Factor IX is separated from plasma hyaluronan binding protease thereby decreasing the extent of Factor IX degradation.

14. The method of claim 13, further comprising an affinity chromatography purification step.

15. The method of claim 14, wherein said affinity chromatography purification step is monoclonal antibody chromatography.

16. The method of claim 13, further comprising a virus reduction step.

17. The method of claim 16, wherein said virus reduction step comprises a pasteurization or nanofiltration step.

18. The method of claim 13, wherein said salt is NaCl or LiCl.
